(19) United States
(12) Patent Application Publication
(10) Pub. No.: US 2002/0065245 A1
(43) Pub. Date: May 30, 2002

(54) Sweetener

(76) Inventor: Louis Jean Hilda Brouwers, Lier (BE)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/844,936

(22) Filed: Apr. 27, 2001

Related U.S. Application Data
(63) Continuation-in-part of application No. PCT/BE99/00137, filed on Oct. 27, 1999.

(30) Foreign Application Priority Data
Oct. 27, 1998 (BE) 98007700

(51) Int. Cl. A61K 31/715; A23G 3/00

(52) U.S. Cl. 514/54; 426/658

(57) ABSTRACT

The invention relates to a novel composition useful for sweetening a food product or a medicine, said composition containing 20-99.9% (v/w) of α-Gluco-oligo-saccharides and 0.1-80% (v/w) of glycosides from steviol.

### REFERENCE TO RELATED APPLICATIONS

This is a continuation-in-part of PCT application PCT/BE99/00137, filed Oct. 27, 1999, which designated the United States and which claimed priority to Belgian application number 98007700, filed Oct. 27 1998.

### FIELD OF THE INVENTION

The invention relates to a novel composition useful for sweetening a food product or a medicine, said composition containing 20-99.9% (v/w) of α-Gluco-oligo-saccharides and 0.1-80% (v/w) of glycosides from steviol.

### BACKGROUND OF THE INVENTION

α-Gluco-oligo-saccharides are comprised of molecules of glucose. Glucose is one of the components of sugar and constitutes a basic component of starch and cellulose. Glucose is the most common sugar in nature.

α-Gluco-oligo-saccharides are “slow” sugars, which on the one hand supply a lot of energy, but on the other hand are often insufficiently absorbed by the digestive system. (α-Gluco-oligo-saccharides have a prebiotic effect on the enzymatic activity of both the intestinal and the cutaneous flora and restore the natural operation thereof. α-Gluco-oligo-saccharides are prebiotic, as they essentially end up in the intestines in an unabsorbed way, where they form a preferential substrate to the bacterial strains in the large intestine, which stimulates the metabolism of the flora near to the large intestine.

α-Gluco-oligo-saccharides have a structure analogous to the complex sugars, which to a high degree are not absorbed during stomach digestion and so end up in the small and large intestines in an undigested form. In the intestinal system, especially in the large intestine, these sugars are consumed by bacteria, which stimulates the metabolism and production of propionate and butyrate without stimulating the production of gas or lactic acid. α-Gluco-oligo-saccharides are therefore an important part of the digestion of complex sugars and benefit digestion. This means that an energy releasing and detoxifying effect will result, without the accompanying drawbacks, e.g. the irregular passage of unfinished digestion in the formation of gas. The α-Gluco-oligo-saccharides thereby leave the intestinal ecosystem intact, stimulate the renewal mechanism on the intestinal wall, and contribute to the reinforcement of the natural defense mechanism. The disadvantages associated with α-Gluco-oligo-saccharides are that the sweetening is insufficient to be used in many food products.

### SUMMARY OF THE INVENTION

The invention relates to the sweetening of α-Gluco-oligo-saccharides with an glycoside of steviol. The glycoside of steviol preferably is steviose. This composition according to the invention has synergetic qualities resulting from an optimal control of the sweetness and the volume of the obtained composition.

### DETAILED DESCRIPTION OF THE PREFERRED EMBODIMENT

Stevioside has a strongly sweet taste, which nevertheless also is accompanied by a bitter aftertaste. This gives stevioside, as such, an undesirable taste. The combination of stevioside and α-Gluco-oligo-saccharides provides a composition with an improved taste and improved digestive qualities. The composition according to the invention additionally is a low in calories, safe for use by diabetics, and water-soluble.

Another advantage of the new composition according to the invention is the stability of the final product, which is heat-resistant and has an adjustable sweetening proportion per volume-unity.

Side effects of the glycoside of steviol, such as diarrhea and gas formation, are removed to a high degree by the composition according to the invention. The composition according to the invention further reduces dental caries and is an ideal composition which in case of stress-symptoms can be administered without drawbacks as a complete stomach digestion is not necessary.

The simple fragmentation, for instance by grinding and mixing, of parts of the Stevia rebaudiana Bertoni plant makes the isolation of sugar contained by the plant no longer necessary. The Stevia plant contains six kinds of sweet glucosides, for instance stevioside, which is about 300 times sweeter than sucrose and reboudioside.

The α-Gluco-oligo-saccharides are preferably derived from enzymatic glucosyl transferase synthesis, starting from sucrose and maltose using a bacterial strain of Leuconostoc mesenteriodes. Such a method is disclosed in EP-A-0 325 872. The α-Gluco-oligo-saccharides can be part of the Bioecolians, manufactured by the firm and being composed of 85-100% (v/w) α-Gluco-oligo-saccharides, 0-5% (v/w) glucose, 0-4% (v/w) fructose, 0-5% water and 0-2% (v/w) of a rest product.

In particular, the α-Gluco-oligo-saccharides in Bioecolians as defined in EP-A-0 325 872 denotes a sugar compound having the general formula: (O-alpha-D-glucopyranosyl-(1→2)ₘ (O-alpha-glucopyranosyl-(1→6)ₙA, wherein A is a glucose acceptor sugar moiety chosen from maltose and methyl alpha-glucoside, m is from 1 to 3, n is from 1 to 10, the position of the alpha (1→2) glucosidic bond(s) being arbitrary.”

With regard to the product Bioecolians the in vitro tests with different bacterial strains being found in the large intestinesprove that Bioecolians is only absorbed by bacterial strains found in the large intestine, e.g. Bacteriodes, Lactobacilles, and bifidogerms, and is not absorbed by pathological carriers in the large intestine, such as Salmonella, staphilococces, Clostrides, and Colibacilles, which provide a selective effect to Bioecolians. Six week in vivo tests, in which a concentration of 4% Bioecolians was administered to rats provided with a human intestinal flora, proved that Bioecolians, in comparison with the control group, where no Bioecolians was administered:

did not disturb the bacteriologic ecosystem in the intestine,

stimulated the enzymatic mechanisms of this ecosystem,

improved the digestion of complex sugars,

stimulated the production of proprionate and butyrate, and

reduced the production of hydrogen gas.

So, Bioecolians has a positive influence on the digestive mechanism and on the production of components stimulating the health, without changing the intestinal ecosysytem. By the selective effect Bioecolians stimulates the natural defense mechanism.

| | | | | BIO- | |
|---|---|---|---|---|---|
| | | CONTROL | | ECOLIANS ® | |
| | Parameters | food | | food | |
| Composition | Bacteroides (log 10/g) | 9.6 | | 9.1 | |
| of the | Bifidobacteria (log 10/g) | 7.4 | | 7.0 | |
| intestinal | | | | | |
| flora | | | | | |
| Enzymatic | α Glucosidase (IU/g) | 0.9 | | 1.5 | |
| activity | β Glucosidase (IU/g) | 0.16 | | 0.50 | |
| of the | α Galactosidase (IU/g) | 1.9 | | 4.0 | |
| intestinal | | | | | |
| flora | | | | | |
| Production | Propionata (μmoles/g) | 6.8 | | 13.8 | |
| digestive gas | Butyrata (μmol/g) | 4.7 | | 6.3 | |
| Cholesterol- | Cholestrol (mg 100 ml) | 120 | | 66 | |
| emia | | | | | |
| Gas | Hydrogen (μmoles) | 13 | | 6 | |
| Body weight | Weight after 6 weeks | 335 ± 20 | g | 335 ± 20 | g |

The biggest drawback of the product Bioecolians is that during the enzymatic production process the sweetening effect fades. The sweetening of the α-Gluco-oligo-saccharides of steviol provides a suitable sweetening of the α-gluco-oligo-saccharides with glycosides or to the Bioecolians product with glycosides of steviol, the drawbacks of the glycosides of steviol, like flatulence or the bitter aftertaste, being nullified.

A composition according to the invention is also preferably diluted with a sugar having a low sweetening capacity, chosen out of the group including inuline, fructo-oligosaccharides, lactitol, maltitol, mannotol, sorbitol, erythritol, galactitol, isomaltulose, polyglucose, polymaltose, carboxymethylcellulose, carboxyethylcellulose, arabinogalactan, microcrystalline cellulose, and dextrose. Inuline, in particular, is a very suitable sugar for this purpose. Inuline is additionally very common, which reduces the cost of a diluted composition according to the invention. The composition, diluted or not, according to the invention is ideal for food products or medicine preparations. When the composition is added to a food product in an amount of 87% (v/w), the storage life of such a food product was extended considerably without causing mold or any other side effect. Food, such as soft drinks, pies, sweets, and jam, are products which are suitable to be sweetened with a composition according to the invention. The composition according to the invention also reduces the amount of cholesterol. A method of treating cholesterol in a mammal comprising administering a therapeutically effective amount of the composition is an additional embodiment of the invention.

Another set of experiments will be described hereunder.

Based on literature, the properties of the α-Gluco-oligo-saccharides (GOS) compound Bioecolians, it was proposed for the first time by Mr. L. Brouwers that a combination of extracts for the plant Stevia rebaudiana Bertoni (family Asteraceae) containing the highly sweet tasting stevioside and rebaudioside A derivatives combined the advantages of both products.

Bioecolians was obtained from Solabia (France, Ref. 361013, lot. 7L254) and is freely available. Extracts from Stevia rebaudiana were used, either in powder form or as a tincture containing water and alcohol.

It was found in experiments on the effect of Stevia on the chemical stability of Bioecolians that the Bioecolians is a highly stable product, not degradable by a number of intestinal enzymes. It is therefore not to be expected that the addition of Stevia extracts will in any way affect the stability of the GOS.

The effect of Bioecolians and Stevia extracts on three principal enzymes of intestinal sugar metabolism have been tested. The studies were performed in vitro, by measuring the enzyme activities with the natural substrates and in the presence of varying concentrations (5 and 30%) of a Bioecolians/Stevia composition containing 13.5% (v/w) Stevia.

No statistically significant changes were found in the activities of β-glucosides and β-galactosideas, indicating that a combination of Bioecolians and Stevia with these enzymes does not affect the specificity and/or maximal activity of these enzymes. In the case of α-glucosidase a small decrease in the enzyme's affinity constant (K_{M}) was apparently detected, indicating a competitive-type inhibition. It thus seemed possible that the enzyme used either Bioecolians or a compound in the Stevia extract as a substrate.

From these results it is apparent that Bioecolians and Stevia have no pronounced effect on the activity of principal sugar-metabolizing enzymes.

In experiments of effects on the growth of bacteria of the intestinal flora it was proven that bacterial cultures did not show significant growth inhibitions.

Bioecolians is considered to have a cholesterolemic effect.

Sensorical analyses were performed. It was found that a concentration of (13.5+/−8.0)% (v/w) Stevia (tincture) in Biolecolians gave a sweetening effect comparable to the sweetness of an identical amount of white sugar. Sensorical (taste) analysis indicates that a composition of Bioecolians containing 10 to 20% (v/w) of a Stevia tincture results in a similar subjective sweetness compared to an equal mass of commercial white sugar.

From the above experiments, it is clear that the novel composition according to the invention improves the taste and the volume effect, which is in particular useful for food preparation while maintaining an improved effect on the growth of bacteria of the intestinal flora.

Further a highly efficient control on the ratio of caloric value/sweetness can be obtained, resulting in a sugar product useful for diabetics.

What is claimed is:

1. A composition for sweetening a food product or a medicine comprising 20-99.9% (v/w) α-gluco-oligo-saccharides and 0.1-80% (v/w) glycosides of steviol.

2. A composition according to claim 1, wherein the glycoside of steviol is stevioside.

3. A composition according to claim 1, wherein the glycoside of steviol is derived from Stevia rebaudiana Bertoni.

4. A composition according to claim 1, characterized in that the α-gluco-oligo-saccharides are derived from an enzymatic glycosyl transferase synthesis, wherein sucrose and maltose are converted to α-gluco-oligo-saccharides by a bacterial strain Leuconostoc mesenteroides.

5. A composition according to claim 1 wherein a Stevia tincture is added to said composition in a concentration of 13.5+/−8% (v/w) in Bioecolians.

6. A composition according to claim 1, wherein the α-gluco-oligo-saccharides has been added to the composition under the product Bioecolians, in particular having the general formula: (O-alpha-D-glucopyranosyl-(1→2)ₘ (O-alpha-glucopyranosyl-(1→6))ₙA, the product consisting of 85-100% (v/w) α-gluco-oligo-saccharide, 0.5% (v/w) glucose, 0-4% (v/w) fructose, 0-5% water, and 0-2% (v/w) from a rest product.

7. A composition according to claim 1 further comprising a sugar component with a low sweetening capacity, selected from the group consisting of inuline, fruco-oligosacharides, lactitol, maltitol, mannitol, sorbitol, eryhritol, galactitol, isomaltulose, polyglucose, polymaltose, carboxymethycellulose, carboxyethylcellulose, arabinogalactan, and microcrystalline cellulose dextrose.

8. A composition according to claim 7, wherein the sugar component is inuline.

9. A food product, such as a food or medicine preparation, comprising a composition according to claim 1.

10. A food product according to claim 9, wherein said composition is at a concentration of 87% (v/w).

11. A pharmaceutical formulation comprising a composition according to claim 1 and a pharmaceutically acceptable carrier or diluent.

12. A method of treating cholesterol in a mammal comprising administering a therapeutically effective amount of the composition according to claim 1.
